(19) United States
(12) Patent Application Publication
(10) Pub. No.: US 2019/0151155 A1
(43) Pub. Date: May 23, 2019

(54) WOUND DRESSING SYSTEM

(71) Applicant: 3M INNOVATIVE PROPERTIES COMPANY, St. Paul, MN (US)

(72) Inventor: Florian Bonn, Leverkusen (DE)

(21) Appl. No.: 15/571,373

(22) PCT Filed: May 10, 2016
(86) PCT No.: PCT/IB2016/052661

§ 371 Date: Nov. 2, 2017

(30) Foreign Application Priority Data
May 11, 2015 (EP) 15167090.8

(51) Int. Cl. A61F 13/00; A61M 3/02; A61M 1/00

(52) U.S. Cl. CPC A61F 13/00068; A61M 3/025; A61M 3/0283; A61M 1/0084; A61M 1/0088; A61M 1/0058

(57) ABSTRACT

The invention relates to a wound dressing system for treating a wound, comprising at least a first and a second sheet-like capillary membrane system,—wherein the first and the second sheet-like capillary membrane systems are in each case connected to at least one supply line, so that fluids, media, gases, and/or other substances can be guided through the supply line and the respective capillary membrane system. According to the invention, the wound dressing system further comprises—a first removal container which is detachably connected via a line path, which comprises at least one of the supply lines of the first capillary membrane system, to the first capillary membrane system, and via which a rinsing fluid can be supplied to said first capillary membrane system,—a second removal container which is detachably connected via a line path, which comprises at least one of the supply lines of the second capillary membrane system, to the second capillary membrane system, and via which a treatment solution can be supplied to said second capillary membrane system, and—a drainage system, which can be coupled to a vacuum unit and via which fluids can be discharged from the wound to be treated.

The invention relates to a wound dressing system for introduction into a wound and/or for placement onto a skin wound and under a wound dressing, comprising at least a first and a second sheet-like capillary membrane system, in which the first and the second sheet-like capillary membrane systems are in each case connected to at least one supply line such that fluids, media, gases, and/or other substances can be supplied through the supply line and the respective capillary membrane system.

The goal of modern wound dressing is to obtain a moist wound environment that promotes ongoing processes during wound healing. Depending upon the stage of healing, modern, active wound dressings must therefore be capable of keeping the wound moist for an improved exchange of fluids/substances, for the supply of factors/medicines, and/or for improved removal of fluid/secretion/substances from the wound or supply to the wound. Applications include the use of such wound dressing systems in a soft-tissue wound, in an abdominal wound, and on a skin wound.

A method and a device for the removal of secretion and/or exudate from wounds is commercially known as the V.A.C® Therapy System (from KCI, USA). With this system, there is an alternating placement of fluid into the wound and, subsequently, also an alternating and thus non-continuous removal of fluid from the wound. A foam material placed into the wound, which exerts forces onto the wound under a vacuum, is intended to promote wound healing with this system.

DE 10 2006 042 732 describes a capillary membrane system for wound treatment in which the wound is perfused and supplied with at least one common supply line and at least one common drainage line in the sense of the flow of a capillary bed by means of a hollow fiber membrane arrangement comprising up to 1000 hollow fibers, and which should make possible perfusion with an antibiotic as well as growth factor. In doing so, a uniform distribution of substances with continuous perfusion, while also creating a moderate vacuum, should be made possible. DE 10 2006 042 732 states that further capillary membrane systems are advantageous for optimal supply and removal.

Even though progress has been made already in wound treatment with the capillary membrane systems described in DE 10 2006 042 732, there is still a need for simple and efficient wound dressing systems with which, on one hand, the processes necessary for wound treatment, such as rinsing and disinfecting, can be carried out without removing the bandage, but which also, if necessary, enable nourishment, exchange of electrolytes, and/or detoxification, or even the supply of growth factors or a supply of antibiotics, and which enable simple and safe handling.

Often, it is desirable to have a suitable wound dressing system that, on one hand, enables efficient cleaning of the wound, but also, on the other hand, enables the targeted application of effective substances into the wound. Therefore, the object of the present invention is to provide such a type of wound dressing system.

The object is achieved by a wound dressing system for treating a wound, which comprises at least a first and a second sheet-like capillary membrane system
wherein the first and the second sheet-like capillary membrane systems are in each case connected to at least one supply line, such that fluids, media, gases, and/or other substances can be supplied through the supply line and the respective capillary membrane system,

wherein the wound dressing system further comprises
a first removal container with a drain, which is detachably connected to the first capillary membrane system via a first drainage line, which comprises at least one of the supply lines of the first capillary membrane system, and by means of which a rinsing fluid can be supplied to the first capillary membrane system,
a second removal container with a drain, which is detachably connected to the second capillary membrane system via a second drainage line, which comprises at least one of the supply lines of the second capillary membrane system, and by means of which a treatment solution can be supplied to the second capillary membrane system, as well as
a drainage system, which can be coupled to a vacuum unit and by means of which fluids can be discharged from the wound to be treated.

Nearly homogenous dressing of a wound is possible by means of such a wound dressing system, in which the configuration of the wound dressing system simultaneously enables safe and easy treatment. In use, the wound dressing system can also be placed into the wound and, for example, be covered using semi-occlusive transparent film in order to protect the wound from drying out or from infections.

The supply lines to the capillary membrane systems then exit from the wound area to the outside from underneath the film and are connected to the first and/or the second removal container outside of the wound area. The drainage system may be coupled to a vacuum unit, for example, via a suitable tube that is stable under vacuum, which likewise exits from the wound area.

In this case, the first or the second sheet-like capillary membrane system consists of one single capillary membrane, which is arranged in a meandering shape. With this embodiment, at least one of the ends of the meandering capillary membrane is open and connected to a supply line. The at least one capillary membrane system may, however, comprise multiple meandering capillary membranes as well, which together with their ends feed into a common supply line. Preferably, the first and/or the second capillary membrane system comprises a plurality of capillary membranes arranged parallel to one another.

The capillary membranes, arranged parallel to one another, of a respective capillary membrane system are embedded with at least one of their ends at their outer periphery so as to be impermeable to fluids in the wall of a supply line, such that a fluid connection exists between the lumen of the supply line and the lumen of the capillary membranes, and fluids, media, gases, and/or other substances can be supplied through the supply line and the at least one capillary membrane system. The embedding may take place, for example, with a curable silicone material, with a polyurethane resin, or an epoxide resin. Preferably, curable silicone materials are used, due to their better flexibility. In the event that the capillary membranes are only embedded with one of their ends in a supply line, the other, opposite end of the capillary membranes is closed—for example, through welding or bonding. The capillary membranes may also be open at both of their ends, and both of these ends may be embedded on one side of the arrangement in one single supply line, in which the capillary membranes are then formed at their free end in the shape of a U and are thereby closed there. In these cases, the capillary membranes are operated in dead-end mode.

Particularly with broader wound dressing systems, embodiment [sic] of the capillary membrane systems with capillary membranes arranged parallel to one another are advantageous, in which the capillary membranes are open at both ends and each embedded in one supply line, in which the supply lines are then preferably located at opposite sides of the respective sheet-like capillary membrane system. In this case as well, the embedding is executed such that the capillary membranes are embedded so as to be impermeable to fluids at their outer periphery, and a fluid connection exists between the lumen of the respective supply line and the lumen of the capillary membranes. Such a type of embodiment with two supply lines enables supply and/or removal in cross-flow mode by means of the at least one capillary membrane system. The design of the at least one capillary membrane system with two supply lines may also be useful as well with respect to good homogeneity of the supply and/or removal by means of the surface of the wound—particularly, with broader meshes and/or wound dressing systems. However, an embodiment with two supply lines also enables simultaneous or even alternating supply of a wound with different media by means of the same capillary membrane system.

The diameter of the supply lines is primarily based upon the outer diameter of the capillary membranes embedded in them. Thus, the at least one supply line preferably has an inner diameter ranging from 0.1 to 10 mm. It is likewise preferable when the wall thickness ranges from 0.1 to 5 mm. In the event that a supply line with a non-circular cross-section is used, the equivalent diameter d=4A/U of the inner cross-section is applied as the inner diameter, where A is the surface area of the inner cross-section and U is its circumference. For example, the supply line may also have an oval or approximately square or rectangular inner cross-section. Silicone tubes, for example, through the wall of which the capillary membranes pass and to which they are bonded, have proven to be suitable for the supply lines. Preferably, the supply lines are formed from a flexible silicone tube. The embedding or the bonding to the wall of the supply line may take place by means of customary adhesives such as, for example, by means of curable silicone materials, polyurethane resins, or an epoxide resins [sic].

In an advantageous embodiment, the first and/or the second capillary membrane system is formed as a capillary membrane mesh made of capillary membranes arranged parallel to one another, in which the capillary membranes in the mesh are connected to one another by means of connecting elements in the mesh, which extend at intervals to one another and parallel to one another, and are maintained at intervals to one another by means of the connecting elements. The connecting elements may extend transversely with respect to the capillary membranes arranged parallel to one another, or even at a different angle. In doing so, the connecting elements make contact with the capillary membranes at their outer periphery, or they wrap around them. The connecting elements do not have any closed flow channels along their longitudinal extension; consequently, fluids cannot flow through along their longitudinal extension. The connecting elements may be adhesive strips or, for example, string-like elements made of a silicone material. In a preferred embodiment, the capillary membranes are connected to a mesh by means of yarn-like connecting elements. Multifilament textile yarns are especially preferred as the connecting elements. Multifilament polyester yarns, polypropylene yarns, or polytetrafluoroethylene yarns have proven especially useful as multifilament textile yarns. Hydrophilic yarns, preferably made of polyester, are most suitable.

In a preferred embodiment, the capillary membrane mesh may be an interwoven mesh. With such interwoven meshes, the capillary membranes and the connecting threads are interwoven with one another, and the capillary membranes extend transversely with respect to the extension direction of the capillary membrane mesh. The length of the capillary membranes is determined by the mesh width. In another preferred embodiment, the capillary membrane mesh may be a web mesh. With such web meshes, the capillary membranes and the connecting threads are interwoven with one another. The capillary membranes in this case extend in the extension direction or movement direction of the capillary membrane mesh, and the textile threads extend transversely thereto. Capillary membrane interwoven meshes and web meshes, as well as the possibilities for the production thereof, are described, for example, in DE 38 39 567, DE 43 08 850, and in EP 0 442 147. Particularly by means of knitting technology, meshes can be produced in a simple manner, in which the capillary membranes are formed in a U-shape at their free ends, and are closed there. Such types of meshes may be produced though meandering placement of a capillary membrane in strands that are parallel to one another, which are connected to one another through the interwoven threads. In doing so, after completion of the interwoven mesh, the U-shaped ends are separated on at least one side of the interwoven mesh, and the resulting open ends of the capillary membranes are then embedded in a supply line. In the event that the U-shaped ends are separated on both side [sic] of the interwoven mesh, the resulting opposite open ends may each be embedded in supply lines.

In a preferred design, the capillary membranes are positioned within the mesh at such a density that the distance between the capillary membranes in the mesh is 1 to 10 times the outer diameter of the capillary membranes, in which the distance is measured from the longitudinal axes of the capillary membranes. In this case, meshes are preferred in which the distance between the capillary membranes in the mash is 1.05 to 6 times the outer diameter of the capillary membranes. It is especially preferable when the distances between the capillary membranes in the mesh range from 1.05 to 3 times the outer diameter of the capillary membranes. In a further, especially preferred embodiment, distances between the capillary membranes in the mesh are more than 1.5 times the outer diameter of the capillary membranes. It has been found that a reliable separation of the capillary membranes from one another can be achieved with this.

At the same time, it may be important with respect to good homogenous dressing of the wound to be treated that the capillary membranes in the capillary membrane systems be connected to a mesh by means of multiple connecting elements, which extend at intervals to one another and parallel to one another, and maintained at intervals to one another by means of the connecting elements, and that the connecting elements be at a defined interval with respect to one another, which is preferably in a range of from 1 to 50 mm, in which an interval ranging from 3 to 20 mm is especially preferable, and one ranging from 4 to 6 mm is most suitable. It has, namely, been shown that the contact points between the capillary membranes and the connecting elements, e.g., with supply of the wound to be treated—for example, with a treatment solution or a nutrient solution—significantly promote distribution of the fluid over the surface area of the arrangement of the capillary membranes. Thus, it has been observed with the placement of such capillary membrane systems onto wounds to be treated that the contact points facilitate the exiting of fluid from the capillary membranes.

The capillary membranes of the capillary membrane systems preferably have an outer diameter ranging from 200 to 1500 μm. Capillary membranes having a wall thickness ranging from 20 to 400 μm are also advantageous, in which their outer diameters may preferably be in the aforementioned ranges.

With the previous wound dressing system, the first and the second capillary membrane systems are designed for the supply and removal, respectively, of fluid media. In order to then ensure uniform dressing of the wound to be treated, in a preferred embodiment, the capillary membranes have high permeability to fluids. Preferably, here, the transmembrane flow for water of the capillary membranes ranges from 0.1 to 500 cm³/(min·cm² MPa) (from 0.01 to 50 mL/(min·cm² bar)).

The capillary membranes of the first and/or second capillary membrane system are preferably impermeable to bacteria. This can ensure that no bacteria reach the wound due to the supply of rinsing fluid and/or treatment solution. In this, impermeability to bacteria within the scope of this invention is understood to mean that the capillary membranes have a nominal pore [size] of 0.2 μm. Preferably, the capillary membranes of the first and/or of the second capillary membrane system thus have a nominal pore [size] of 0.2 μm. The nominal pore [size] here is defined by means of the retention capacity of the membrane compared to the specific microorganisms. Thus, for example, a membrane with a nominal pore [size] of 0.2 μm retains bacteria of the genus Brevundimonas diminuta, but also bacteria of the genus Serratia marcescens, for which a membrane with a nominal pore [size] of 0.45 μm would have been sufficient. The tests and/or the determination of the nominal pore sizes is described, for example, in HIMA Regulation No. 3, Vol. 4, 1982 (Health Industry Manufacturers Association).

Essentially all of the organic polymers known in the prior art, which are suitable for forming capillary membranes, are suitable as the materials for the capillary membranes, but these polymers must have good biocompatibility. Furthermore, it is also necessary for the membrane polymer to enable sterilization of the wound dressing system—for example, by means of vapor sterilization, sterilization by means of y-radiation, or sterilization by means of ethylene oxide. In doing so, the organic polymers may be natural polymers, or polymers that have been produced in synthetic manners. Natural polymers are particularly those based on cellulosic polymers, which also includes polymers that have been subjected to so-called polymer-analogous reactions. Examples of polymers based on cellulose are those made from regenerated cellulose, cellulose acetate, or modified cellulose such as, for example, cellulose ester, cellulose ether, cellulose modified with benzyl groups (benzyl cellulose), or cellulose modified with diethyl amino ethyl, or mixtures of these cellulosic polymers. Furthermore, polymers based on chitin or chitosan may be used.

Polymers produced in synthetic ways, i.e., as synthetic polymers, may include those that consist of polyolefins, polyamides, polyacrylonitriles, polycarbonates, polyesters, or sulfone polymers, as well as the modifications, blends, mixtures, or copolymers of these polymers obtained therefrom. Preferably, those that are based on sulfone polymers such as, in particular, polysulfone or polyethersulfone are used. Further polymers such as, for example, polyethylene oxide, polyhydroxy ether, polyethylene glycol, polyvinyl alcohol, or polycaprolactone can be mixed into the synthetic polymers as additives. The capillary membranes may furthermore have a coating with an additive. Such capillary membranes preferably contain a hydrophilization agent, such as polyvinylpyrrolidone or even hydrophilic modifications of these polymers.

With respect to certain applications, the capillary membranes may be modified, e.g., by linking functional groups, or coated, for example, with heparin or an antibiotic or several antibiotics.

The sheet-like capillary membrane systems may have any shape in their sheet-like extension. In the event of capillary membrane systems comprising capillary membranes parallel to one another, the capillary membrane systems have a square or rectangular shape, in the simplest design. However, with systems, for example, in which the capillary membranes are only embedded in a supply line at one of their ends, it is possible for a bow-shaped contour to be formed, for example, due to correspondingly adapted sealing of the free, sealed ends of the capillary membranes parallel to one another. It is likewise possible for the arrangement comprising capillary membranes parallel to one another to also have, for example, a trapezoidal contour.

The present wound dressing system may also have further components such as, for example, at least one further arrangement of capillary membranes. The capillary membranes of the further arrangement may, for example, be membranes for oxygenation, i.e., membranes which enable the supply of oxygen to the wound. Such types of membranes are disclosed, for example, in EP-A-1 144 096, EP-A-0 299 381, or DE-A-28 33 493. A combination with further sheet-like systems and/or arrangements of semi-permeable capillary membranes or fluid-impermeable capillaries is also possible, by means of which, for example, temperature control or pH-value regulation may take place. In doing so, the respective capillary membrane systems and, possibly, further sheet-like systems of semi-permeable capillary membranes or fluid-impermeable capillaries may be placed on top of one another. However, it is also possible for the capillary membranes of different capillary membrane systems, for example, to be connected in one mesh, in which the ends of the different capillary membranes are embedded in different supply lines, which are preferably arranged at opposite sides of the mesh. Such meshes may be maintained, for example, through interweaving of capillary membranes arranged offset from one another and placed in a meandering shape, in which the U-shaped deflections of the capillary membranes are at different positions over the breadth of the mesh. By cutting the U-shaped deflections to the outside, the capillary membranes are opened on only one side of the mesh and can be embedded therein in a supply line.

According to the invention, in addition to the first and the second capillary membrane systems, the wound dressing system comprises a drainage system, by means of which drainage, for example, of the rinsing fluid or of exudate from the wound is possible. In one embodiment, the wound dressing system may have a suction sponge, which has a suction line for the rinsing fluid and/or for the exudate. The drainage system may also be formed as a further capillary membrane system. Preferably, the drainage system, however, comprises at least one drainage catheter, e.g., in the form of a piece of tubing, for example, made of silicone material or a small tube. Such a drainage catheter may have perforations in its wall, by means of which fluids can be suctioned from the wound after connection of the drainage catheter to a vacuum unit. The at least one drainage catheter preferably has an inner diameter ranging from 0.1 to 15 mm and a wall thickness ranging from 0.1 to 3 mm. The drainage catheter may also have a non-circular cross-section. In this case, the equivalent diameter d_{D}=4A_{D}/U_{D} of the inner cross-section is applied as the inner diameter, where A_{D} is the surface area of the inner cross-section of the drainage catheter, and U_{D} is its circumference.

In one embodiment, the wound dressing system furthermore may comprise a bag-type wound dressing, in which the outer edge of the bag-type wound dressing is closed and has a top side, a bottom side, and a bag interior, in which the bottom side and the top side each are formed from a flat material and the bottom side is permeable to fluids, and in which the first and the second capillary membrane systems are arranged in the interior of the bag. In this case, the connection of the supply lines to the respective collection containers is outside of the bag-type wound dressing.

The bag-type wound dressing with the capillary membrane systems contained therein can thus be placed into a wound to be treated such that the bottom side is in contact with the wound. The capillary membrane systems can be used to supply the desired fluids to the wound, with the fluids being distributed in the bag after exiting the capillary membranes and dispensed to the wound via the semi-permeable, bottom side of the bag.

The bag-type wound dressing may preferably be designed such that the connection of the capillary membranes of the capillary membrane systems to the respective supply line may be in the interior of the bag, and the respective supply lines exit from the bag-type wound dressing via passages adapted to be impermeable to fluids at their external cross-section. In a similar manner, the connection of the capillary membranes to the respective supply line may be arranged outside of the bag-type wound dressing on the top side, and the arrangement of the capillary membranes for connecting to the at least one supply line exit from the bag-type wound dressing via a passage adapted so as to be impermeable to fluids.

In the event that the wound dressing system comprises a bag-type wound dressing, in which the first and the second capillary membrane systems are arranged, the capillary membrane systems extend in a sheet-like manner in the bag interior. The dimensions of the capillary membrane systems each result from their external dimensions in the sheet-like extension. Preferably, the first and second capillary membrane systems, with respect to their sheet-like extension, fill the bag interior of the bag-type wound dressing, in its sheet-like extension, to at least 20%, and, especially preferably, to at least 50%. It is especially advantageous when the first and the second capillary membrane systems, with respect to their sheet-like extension, fill the bag interior of the bag-type wound dressing, in its sheet-like extension, to at least 70%, in which fill levels in the range of 90% may also be realized. In doing so, it is advantageous when the first and second capillary membrane systems are arranged in the middle of the bag-type wound dressing.

The bag-type wound dressing may have any contour. However, the contour is preferably round, oval, square, or rectangular. The bottom side and top side of the bag-type wound dressing are bonded to each other on the outer edge or on the outer edges of the wound dressing, for example, through welding or bonding. Silicone strips which are cured are, inter alia, suitable for the bonding. With the rectangular or square bag-type wound dressings, the sheet-like capillary membrane systems arranged therein preferably also have a rectangular or square contour. With round or oval bag-type wound dressings, the capillary membrane systems therein are likewise expediently formed as a square or rectangle, in which the aforementioned dimensions also apply. However, they may also be adapted to the contour of the bag-type wound dressing, for example, through correspondingly adapted sealing of the non-embedded ends of the capillary membranes for capillary membrane systems having only one supply line, such that a bow-shaped contour results at this edge of the capillary membrane systems.

The bottom side of the bag-type wound dressing is permeable to fluids. In this case, the bottom side may consist of a non-woven flat material, a material in the shape of a grid or network, a perforated film, or a semi-permeable, microporous flat membrane. In an advantageous embodiment, the bottom side consists of a non-woven, flat material or a semi-permeable, microporous flat membrane. The bottom side preferably has a permeability to water of at least 0.1 cm³/(min·cm²·MPa) (0.01 mL/(min·cm²·bar)) and, especially preferably, of at least 100 cm³/(min·cm²·MPa) (10 mL/(min·cm²·bar)). A bottom side having a permeability to water of at least 5000 cm³/(min·cm² MPa) (500 mL/(min·cm²·bar)) has proven to be best.

For the intended applications of the wound dressing system, in which the wound is not only supplied with fluid by means of the wound dressing system, but in which fluids are also removed, i.e., discharged, from the wound by means of the drainage system, it is advantageous when openings are provided in the bottom side, in which the openings preferably have a diameter of at least 100 μm. In doing so, diameters of the openings of no more than 10 mm are preferred, and no more than 5 mm are especially preferred. In the event that the bottom side consists of a semi-permeable, microporous flat membrane, it additionally has openings, e.g., in the form of perforations, in an advantageous embodiment. In the event the openings have a non-circular contour, the equivalent diameter d=4A/U of the opening is used as the diameter, where A is the surface area of the respective opening and U is its circumference. The openings may be regularly or irregularly distributed over the surface of the bottom side, in which a regular, homogenous distribution is preferred. In this case, the interval between the openings may range from 1 to 20 mm, measured from the outer edge of the openings.

The bottom and the top sides of the bag-type wound dressing may consist of similar or different materials. While the bottom side is always permeable to fluids, the top side is preferably formed from a fluid-impermeable, preferably film-like material, which is connected to the bottom side on its side edge or side edges so as to be impermeable to fluids. The top side may be a semi-permeable, microporous flat membrane. In this case, the top side, however, has less permeability to fluids than the bottom side, in order to ensure distribution of supplied fluid on the bottom side of the bag-type wound dressing, and thus to the wound, during application. In the event that the bottom side and the top side are the same or similar semi-permeable, microporous flat membrane, the bottom side has perforations.

Essentially the same organic polymers that were previously listed as the polymers for the capillary membranes and that can be processed into flat films or flat membranes are suitable as the materials for the bottom and the top sides of the bag-type wound dressing. Preferably, the bottom and/or top side of the bag-type wound dressing is made up of polyolefins, polyamides, polyacrylonitrile, polycarbonates, polyesters, or sulfone polymers, as well as the modifications, blends, mixtures, or copolymers of these polymers obtained therefrom. Especially preferably, the bottom and top sides comprise sulfone polymers as the material, in which polysulfone or polyethersulfone are most suitable.

In the preferred case that the wound dressing system comprises a bag-type wound dressing, the drainage system, preferably in the form of at least one drainage catheter, exits from the bag-type wound dressing via a passage adapted to be impermeable to fluids and can be coupled to a vacuum unit outside of the bag-type wound dressing in order to establish a vacuum on the inside of the bag during application. The at least one drainage catheter may be a piece of tubing, e.g., made of a silicone material, or a small tube, which is arranged in the interior of the bag of the wound dressing and exits from the wound dressing by means of the passage. At the segment of the at least one drainage catheter located in the interior of the bag-type wound dressing, it preferably has perforations in its wall by means of which fluids such as, for example, exudate, can be suctioned from the wound or from the inside of the bag-type wound dressing and from the wound, after connection of the at least one drainage catheter to a vacuum unit. In a preferred embodiment, the first and second capillary membrane systems of the wound dressing system are identical and form a single supply capillary membrane system, which is connected to at least one supply line, and the first removal container and the second removal container are connected to the supply capillary membrane system by means of the at least one supply line of the supply capillary membrane system. In doing so, the supply capillary membrane system may have two supply lines, which are located at the opposite ends of its capillary membranes. In this case, the first removal container for rinsing fluid and the second removal container with treatment solution, for example, may be connected to different supply lines that are separate from one another. However, it is also possible for the first removal container for rinsing fluid and the second removal container with treatment solution to be connected to only one supply line of the supply capillary membrane system by means of sections of line connected to one another, for example, by means of a T-piece or Y-connector. In this case, designs of the supply capillary membrane system are also possible in which the supply capillary membrane system is connected to only one supply line.

It is preferable for the application when the rinsing fluid from the first removal container and the treatment solution from the second removal container can be supplied separately from one another and alternatingly. To this end, preferably the drainage line between the first removal container and the capillary membrane system connected to the first removal container has a first regulating element allocated to the first removal container. Likewise, the drainage line between the second removal container and the capillary membrane system connected to the second removal container has a regulating element allocated to the second removal container, in which the first and the second regulating elements can be adjusted independently of one another. Depending on the design of the wound dressing system, the regulating elements at the respective removal containers may be placed in the drainage lines between the removal containers and a T-piece or Y-connector connecting the drainage lines of the removal containers or in the respective supply line. In one embodiment, the regulating elements may be a blocking element by means of which the respective drainage line can be opened or closed. However, there can also be regulating elements by means of which the flow through the drainage line can be adjusted to defined values.

In a preferred embodiment, the first and second removal containers are detachably connected to the respective supply line by means of male/female connectors. Especially preferably, the male/female connectors are Luer lock connectors. In this case, a section of tubing, which in turn is connected to the respective supply line, may also be connected to the respective removal container. The connection between the respective removal container and the supply line, i.e., between the removal container and the supply line, between the section of tubing and the supply line, or between the partial sections of the section of tubing, may also be implemented as a sterile welding connection, as can be produced by means of a sterile connector (e.g., TSCD® II Sterile Tubing Welder, Terumo).

In a further advantageous embodiment, the first and/or the second removal container consists of a plurality of first partial removal containers, which are arranged in a parallel circuit. The outlets of the partial removal containers are connected to a partial drainage line, which are connected to the supply line of the first and the second capillary membrane systems, respectively, by means of a connecting element and a connecting line connected thereto. In doing so, the partial drainage lines preferably each have a regulating element, by means of which the respective partial removal containers can be switched on or switched off, or by means of which the flow through the partial drainage line can be adjusted to defined values. Such an embodiment is especially advantageous, for example, when the wound is supposed to be supplied with endogenous serum, for example, which is divided into multiple, partial removal containers as aliquots.

As stated, wound treatments often require phases, in which there is rinsing of the wound alternating with phases in which there is an application of a treatment solution into the wound area. To this end, the present wound treatment system comprises a first removal container for a rinsing fluid and a second removal container for a treatment solution. Normally, the treatment solutions are only supplied to the wound area in small quantities, while larger volumes of rinsing solution are required for sufficient cleaning of the wound and for removal of the waste products. Thus, in a preferred embodiment, the ratio between the volume of the first removal container and the volume of the second removal container is at least 5. Especially preferably, the ratio is at least 10, and, very especially preferably, it is at least 20. The absolute volumes of the first and of the second removal containers depend, inter alia, on the size of the wound to be treated.

Customary fluids may be suitable as the rinsing fluid for wound cleaning. The first removal container preferably contains a sodium chloride solution. Solutions with growth factors, antibiotics, or other medication-containing solutions, solutions for regulating the pH value, or even exogenous or endogenous serum are suitable as the treatment solution. In a preferred embodiment, the second removal container contains a serum.

First and/or second removal containers may be subjected to pressure in a preferred design. In the application, however, it may be sufficient to arrange the first and/or second removal container at a defined vertical height above the wound by means of a suitable retaining device, such that the flow out of the first and/or second removal device and the supply of the rinsing fluid and/or the treatment solution take place under the influence of gravity.

The following measuring methods were used as the basis for characterizing the properties of the capillary membranes and flat membranes used in the wound dressing system.

Transmembrane Flow (Water Permeability) for Capillary Membranes:

A test cell with a defined capillary membrane number and length was produced from the capillary membranes to be tested. For this purpose, the capillary membranes were embedded on both sides with their ends in a polyurethane resin. After the resin was cured, the embedded contents were cut to a length of about 30 mm, in which the lumens of the capillary membranes were opened by the cutting. The capillary lumens in the embedded contents had to be examined for continuity. The free length of the capillary membranes between the embedded contents was typically 120+/−10 mm. The number of capillary membranes had to be measured such that, taking into consideration the free length and the interior diameter of the capillary membranes, a filtration surface of about 30 cm² was provided in the test cell.

The test cell was integrated into a test device, and ultra-filtered and demineralized water, which had been brought to a temperature of 25° C., flowed through it at a defined test pressure (about 0.04 MPa (0.4 bar)). The filtrated water quantity obtained over a measurement time of 2 minutes, i.e., the permeate created during the measurement, was determined gravimetrically or volumetrically. Before start of the measurement, the equipment must be flushed free of air. To determine the TMF, the input and the output pressure in the test device was measured at the test cell. The measurement was conducted at 25° C.

The transmembrane flow TMF was determined according to formula (I)

$$TMF = \frac{{V}_{w}}{Δ t \cdot {A}_{M} \cdot Δ p} [ \frac{ml}{{cm}^{2} \cdot min \cdot bar ( {cm}^{2} \cdot min \cdot bar )} ] \qquad ( I )$$

where:
V_{W}=volume of water [mL] that flowed through the membrane specimen during the measurement time
Δt=measurement time [min]
Δ_{M}=surface area of the membrane specimen [that water] flowed through (typically, 30 cm²)
Δp=set pressure during the measurement [MPa (bar)]

Permeability to Water of the Bottom Side of the Bag-Type Wound Dressing:

Disk-shaped specimens of the flat material to be tested were punched out from the bottom side of the bag-type wound dressing and tensioned in a suitable specimen holder so as to be impermeable to fluids at the periphery, such that a free measurement surface of 17.35 cm² resulted. The specimen holder was in a housing, which could be subjected to the pressurized flow of water. The tensioned specimen was then subjected to a flow of demineralized water, which had been brought to a temperature of 25° C., at a defined pressure between 0.01 and 0.02 MPa (between 0.1 and 0.2 bar). During a measurement time of 60 s, water volumes that flowed through the specimen were determined gravimetrically or volumetrically.

The permeability to water TMF_{W} was determined according to formula (II)

$${TMF}_{w} = \frac{{V}_{w}}{Δ t \cdot {A}_{M} \cdot Δ p} [ \frac{ml}{{cm}^{2} \cdot min \cdot bar ( {cm}^{2} \cdot min \cdot bar )} ] \qquad ( II )$$

where:
V_{W}=volume of water [mL] that flowed through the specimen during the measurement time
Δt=measurement time [min]
Δm=surface area of the specimen [that water] flowed through (17.35 cm²)
Δp=set pressure during the measurement [MPa (bar)]

The invention will be explained by means of the following figures, in which the scope of the invention is not limited by the figures:

The following is shown:

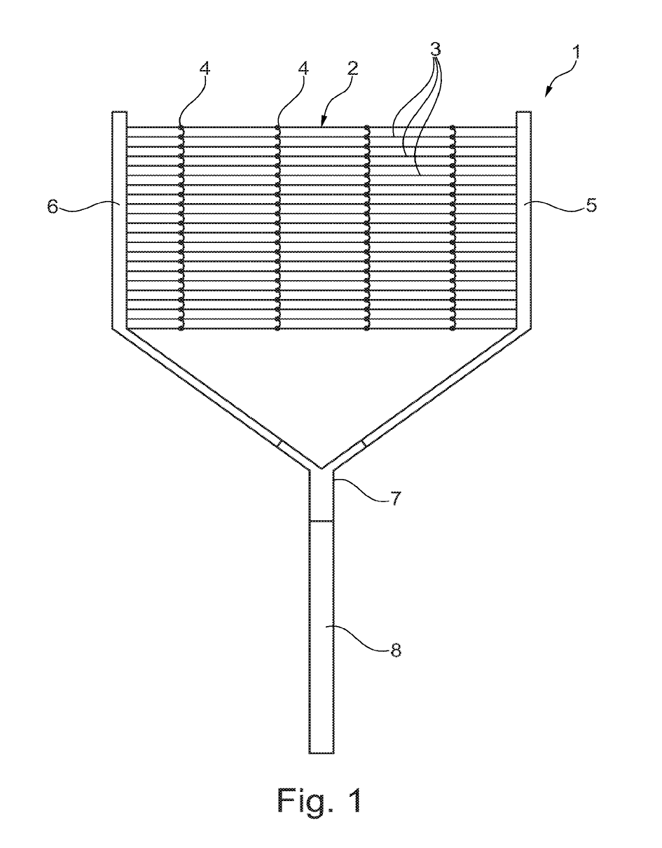

FIG. 1: A capillary membrane system usable in the wound dressing system, with a mesh made of capillary membranes and supply lines at both ends of the mesh;

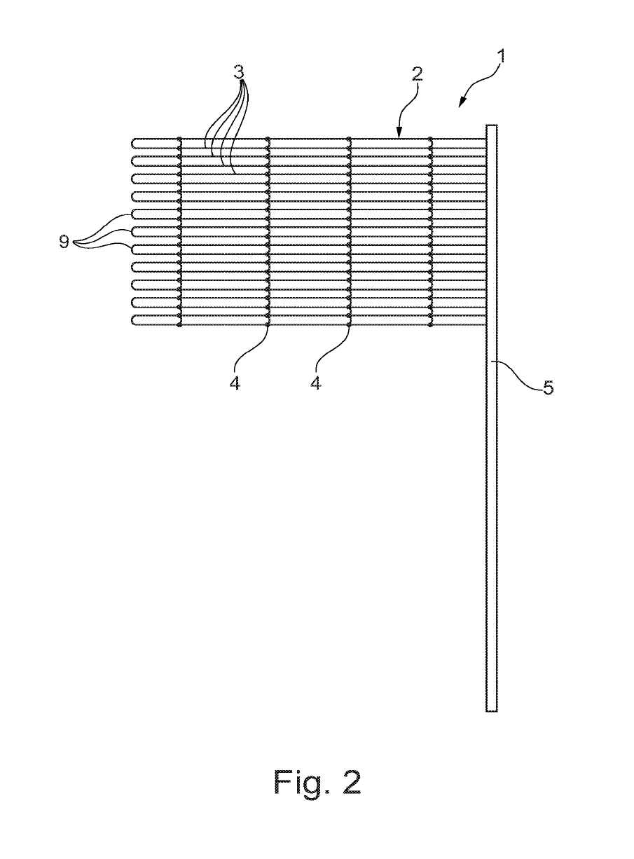

FIG. 2: A capillary membrane system usable in the wound dressing system with a supply line at one of the mesh ends, as well as U-shaped capillary membranes at the opposite mesh end;

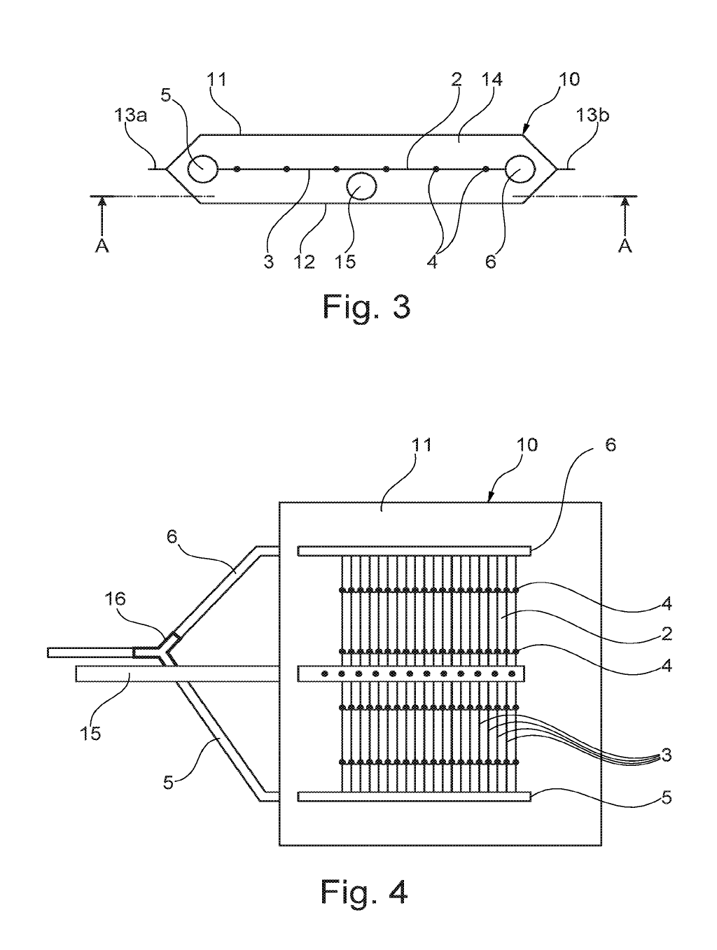

FIG. 3: Cross-section (schematic) through a bag-type wound dressing usable in the wound dressing system;

FIG. 4: Section A-A of the bag-type wound dressing shown in cross-section in FIG. 3;

FIG. 1 shows a top view schematically and not to scale of a capillary membrane system 2 made of capillary membranes 3 usable in the wound dressing system 1 according to the invention. The capillary membranes 3 are connected to a mesh by means of connecting elements 4 extending parallel to one another such that they are arranged parallel to one another and are maintained at an interval with respect to one another. In the present example, the opposite ends of the capillary membranes 3 are embedded in the supply lines 5, 6 such that a fluid connection exists between the lumens of the supply lines 5, 6 and the lumen of the capillary membranes 3. The supply lines 5, 6 are combined into one common line 8 by means of a Y-piece 7. This structure enables the rinsing fluid and/or the treatment solution, which is supplied via the line 8, to be distributed to the supply lines 5, 6 and supplied to the capillary membranes 3 in dead-end mode. The rinsing fluid and/or the treatment solution then flows over the porous, semi-permeable walls of the capillary membranes 3 and exits from them, and is uniformly supplied to the wound by means of the surface of the capillary membrane system 2.

FIG. 2 also schematically shows, in a representation that is not to scale, a wound dressing system 1, in which the capillary membranes 3 are only connected to one supply line 5. The two ends of the capillary membranes are open, and the two ends are embedded in one supply line 5. The free ends 10 of the capillary membranes 3 are in a U-shape at the end of the mesh opposite the supply line 5, and thereby closed there. In this manner, the flow takes place in dead-end mode with the capillary membranes 3 of the capillary membrane system 2 shown in FIG. 3.

FIG. 3 schematically shows a cross-section through a bag-type wound dressing 10, which has a top side 11 and a bottom side 12, which are welded together, e.g., at their edges 13a, 13b, whereby a closed bag interior 14 results. The bag interior 14 here only contains a first capillary membrane system 2, in order to simplify the representation, which comprises capillary membranes 3, which are preferably connected to one another in the form of multifilament yarns and are maintained at an interval with respect to one another by means of connecting elements 4 extending parallel to one another. A second membrane system required according to the present invention may be arranged in one embodiment above or below the shown capillary membrane system 2, in which the supply lines of the first capillary membrane system and of the second capillary membrane system may then exit on the same side or on different sides of the bag-type wound dressing 10.

In the present case, the opposite ends of the capillary membranes 3 feed into supply lines 5, 6 such that fluids, media, gases, and/or other substances can be supplied through the supply lines 5, 6 and the capillary membrane system 2. The supply lines 5, 6 exit through the top side 11 of the bag-type wound dressing 10 (not shown here).

A drainage tube 15, by means of which, for example, exudate collected in the wound can be removed, is arranged below the flat capillary membrane system 2.

FIG. 4 shows the wound dressing system shown in FIG. 3 in a cross-section along line A-A. In principle, this is a top view from a position above the bottom side 12 of the bag-type wound dressing 10 in the direction of the top side 11 of the bag-type wound dressing 10. The capillary membrane system 2, which is composed of capillary membranes 3 parallel to one another, which are connected to one another and maintained at an interval with respect to one another by means of the connecting elements 4, is arranged below the top side 11, i.e., as shown in FIG. 3, between the bottom side 12 and the top side 11. The opposite ends of the capillary membranes 3 are embedded into supply lines 5, 6 such that fluids, media, gases, and/or other substances can be supplied through the supply lines 5, 6 and the capillary membrane system 2. The supply lines 5, 6 exit from the bag-type wound dressing 10 through the top side 11 of the bag-type wound dressing 10 via correspondingly adapted openings in the top side 11 and, in the present example, are combined via a Y-connector 16 outside of the bag-type wound dressing 10. In the present case, the capillary membrane system 2 is thus operated in dead-end mode, i.e., a medium supplied by means of the supply lines 5, 6 is introduced into the capillary membrane system 2 and enters the interior of the bag completely via the walls of the capillary membranes 3.

FIG. 2 also shows the drainage tube 15, which is arranged underneath the capillary membrane system 2. The drainage tube has perforations in its walls such that, for example, exudate collected in the wound can be suctioned and thus removed from the wound via the drainage tube. The drainage tube 15 likewise exits from the bag-type wound dressing 10 via a correspondingly adapted opening in the top side 11 and can be connected, for example, to a vacuum unit (not shown).

1. Wound dressing system for treating a wound, comprising at least a first and a second sheet-like capillary membrane system,
wherein the first and the second sheet-like capillary membrane systems are in each case connected to at least one supply line such that fluids, media, gases, and/or other substances can be supplied through the supply line and the respective capillary membrane system,
wherein the wound dressing system further comprises
a first removal container with a drain, which is detachably connected to the first capillary membrane system via a first drainage line, which comprises at least one of the supply lines of the first capillary membrane system, and by means of which a rinsing fluid can be supplied to the first capillary membrane system,
a second removal container with a drain, which is detachably connected to the second capillary membrane system via a second drainage line, which comprises at least one of the supply lines of the second capillary membrane system, and by means of which a treatment solution can be supplied to the second capillary membrane system, as well as
a drainage system, which can be coupled to a vacuum unit and by means of which fluids can be discharged from the wound to be treated.

2. Wound dressing system according to claim 1, characterized in that the first and/or the second capillary membrane system is formed as a mesh made of capillary membranes arranged parallel to one another, wherein the capillary membranes in the mesh are connected to one another by means of connecting elements in the mesh, which extend at intervals to one another and are parallel to one another, and which are maintained at intervals to one another by means of the connecting elements.

3. Wound dressing system according to claim 2, characterized in that the capillary membranes are connected to a mesh by means of yarn-like connecting elements.

4. Wound dressing system according to claim 1, characterized in that the capillary membranes of the first and/or of the second capillary membrane system are embedded with at least one of their ends in a single supply line.

5. Wound dressing system according to claim 1, characterized in that the first and/or the second capillary membrane system is connected to two supply lines, wherein the capillary membranes of the respective capillary membrane system are embedded with their opposite ends in one single supply line each.

6. Wound dressing system according to claim 1, characterized in that the capillary membranes of the first and/or of the second capillary membrane system have a transmembrane flow for water ranging from 0.1 to 500 cm3/(min·cm2·MPa) (from 0.01 to 50 mL/(min·cm2·bar)).

7. Wound dressing system according to claim 1, characterized in that the capillary membranes of the first and/or of the second capillary membrane system have a nominal pore [size] of 0.2 μm.

8. Wound dressing system according to claim 1, characterized in that it further comprises a bag-type wound dressing, wherein the outer edge of the bag-type wound dressing is closed and has a top side, a bottom side, and a bag interior, wherein the bottom side and the top side are each formed from a flat material and the bottom side is permeable to fluids, and wherein the first and the second capillary membrane systems are arranged in the interior of the bag.

9. Wound dressing system according to claim 8, characterized in that the drainage system is arranged in the interior of the bag and can be coupled to a vacuum unit via a connecting line exiting from the bag-type wound dressing.

10. Wound dressing system according to claim 8, characterized in that the drainage system is a drainage catheter, which exits the bag-type wound dressing via a passage adapted to be sealed against fluids at its cross-section and which can be coupled to a vacuum unit.

11. Wound dressing system according to claim 1, characterized in that the bottom side of the bag-type wound dressing is formed from a non-woven, flat material or a semi-permeable, microporous flat membrane.

12. Wound dressing system according to claim 1, characterized in that the bottom side of the bag-type wound dressing has openings.

13. Wound dressing system according to claim 1, characterized in that the first and second capillary membrane systems are identical and form a single supply capillary membrane system, which is connected to at least one supply line, and that the first removal container and the second removal container are connected to the supply capillary membrane system by means of at least one supply line of the supply capillary membrane system.

14. Wound dressing system according to claim 1, characterized in that the ratio between the volume of the first removal container and the volume of the second removal container is at least 5.

15. Wound dressing system according to claim 1, characterized in that it comprises at least one further arrangement of capillary membranes.
